(19) United States
(12) Patent Application Publication
(10) Pub. No.: US 2021/0046207 A1
(43) Pub. Date: Feb. 18, 2021

(54) METHODS FOR REDUCING ODORS PRODUCED BY TERPENES

(71) Applicant: CENTRAL COAST LAND GROUP, LLC, Solvang, CA (US)

(72) Inventors: Tareq John Moussa DABDOUB, Solvang, CA (US); Atif DABDOUB, Atlanta, GA (US); Pilar Suzann GATES, Solvang, CA (US); Timothy Lanier GRAHAM, Cartersville, GA (US)

(21) Appl. No.: 16/992,420

(22) Filed: Aug. 13, 2020

Related U.S. Application Data
(60) Provisional application No. 62/887,005, filed on Aug. 15, 2019.

(51) Int. Cl. A61L 9/01; C11D 1/29; C11D 3/39

(52) U.S. Cl. CPC A61L 9/01; A61L 2209/21; C11D 3/3942; C11D 1/29

(57) ABSTRACT

Described herein are methods for reducing odors produced from terpenes. The methods involve treating air comprising one or more terpenes in order to reduce the odor. In one aspect, the air comprising the terpenes are circulated through compositions that can convert the terpenes to new chemical species that are not as odorous or possess no odor at all. The methods described herein have numerous applications where terpenes are produced or processed and it is desirable to reduce the odor produced by these compounds.

### CROSS REFERENCE TO RELATED APPLICATIONS

This application claims priority upon U.S. provisional application Ser. No. 62/887,005 filed on Aug. 15, 2019. This application is hereby incorporated by reference in its entirety.

### BACKGROUND

Terpenes are a large and diverse class of organic compounds produced by a variety of plants. They often have a strong odor and may protect the plants that produce them by deterring herbivores and by attracting predators and parasites of herbivores. Terpenes are the primary constituents of the essential oils of many types of plants and flowers. Essential oils are used widely as fragrances in perfumery and traditional medicine, such as aromatherapy.

While terpenes are aromatic, the odor produced from them can range from being a nuisance to posing as a significant health risk. This is particularly relevant with the cultivation of the species Cannabis sativa. The leaves, flowers and stems of Cannabis sativa (i.e, marijuana) and the leaves and flowering tops of the plant (i.e, hemp) possess large amounts of terpenes. With the legalization of marijuana on the rise, the cultivation of Cannabis sativa and subsequent odor abatement will be of environmental significance.

There are currently several techniques for reducing odor produced from the cultivation of Cannabis sativa. One approach involves the use of ozone generators and UV lamps in a greenhouse. However, these approaches can be detrimental to the plants as well as pose a health risk to workers. Another approach involves masking the odors produced by the plants by spraying the plants with a chemical. This approach, however, is not a long-term solution in view of the growing demand for Cannabis sativa. What is needed are more effective and safe methods for reducing odor produced from terpenes.

### SUMMARY

Described herein are methods for reducing odors produced from terpenes. The methods involve treating air comprising one or more terpenes in order to reduce the odor. In one aspect, the air comprising the terpenes are circulated through compositions that can convert the terpenes to new chemical species that are not as odorous or possess no odor at all. The methods described herein have numerous applications where terpenes are produced or processed and it is desirable to reduce the odor produced by these compounds.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below:

FIGS. 1 and 2 show an exemplary implementation of the methods described herein, where the method is used in a greenhouse.

### DETAILED DESCRIPTION

Before the present materials, articles and/or methods are disclosed and described, it is to be understood that the aspects described below are not limited to specific compounds, synthetic methods, or uses, as such may, of course, vary. It is also to be understood that the terminology used herein is for the purpose of describing particular aspects only and is not intended to be limiting.

In the specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “a terpene” includes mixtures of two or more terpenes and the like.

“Optional” or “optionally” means that the subsequently described event or circumstance can or cannot occur, and that the description includes instances where the event or circumstance occurs and instances where it does not. For example, the compositions described herein may optionally contain a defoamer, where the defoamer may or may not be present.

Throughout this specification, unless the context dictates otherwise, the word “comprise,” or variations such as “comprises” or “comprising,” will be understood to imply the inclusion of a stated element, integer, step, or group of elements, integers, or steps, but not the exclusion of any other element, integer, step, or group of elements, integers, or steps.

As used herein, the term “about” is used to provide flexibility to a numerical range endpoint by providing that a given numerical value may be “a little above” or “a little below” the endpoint without affecting the desired result. For purposes of the present disclosure, “about” refers to a range extending from 10% below the numerical value to 10% above the numerical value. For example, if the numerical value is 10, “about 10” means between 9 and 11 inclusive of the endpoints 9 and 11.

As used herein, the term “admixing” is defined as mixing two or more components together so that there is no chemical reaction or physical interaction. The term “admixing” also includes the chemical reaction or physical interaction between the two or more components.

As used herein, “terpene” refers to a class of compounds that consist of one or more isoprene units. Terpenes may be linear (acyclic) or contain rings. A terpene containing oxygen functionality or missing a methyl group is referred to herein as a terpenoid. Terpenoids fall under the class of terpenes as used herein.

As used herein, “hemiterpene” refers to a class of terpenes that consist of isoprene unit and have the molecular formula C₅H₈. Monoterpenes may be linear (acyclic) or contain rings. A hemiterpene containing oxygen functionality is referred to herein as a hemiterpenoid.

As used herein, “monoterpene” refers to a class of terpenes that consist of two isoprene units and have the molecular formula C₁₀H₁₆. Monoterpenes may be linear (acyclic) or contain rings. Examples of monoterpenes include, but are not limited to, myrcene, limonene, carene, sabinene, camphene, and thujene. A monoterpene containing oxygen functionality or missing a methyl group is referred to herein as a monoterpenoid. Examples of monoterpenoids include, but are not limited to, menthol and carvone.

As used herein, “sesquiterpene” refers to a class of terpenes that consist of three isoprene units and have the molecular formula C₁₅H₂₄. Sesquiterpenes may be linear (acyclic) or contain rings. Examples of sesquiterpenes include, but are not limited to, zingiberene, cadinene, cadalane, muurolene, amorphene, and bulgarene. A sesquiterpene containing oxygen functionality or missing a methyl group is referred to herein as a sesquiterpenoid. Examples of sequiterpenoids include, but are not limited to, humulene, farnesenes, and farnesol.

As used herein, “diterpene” refers to a class of terpenes that consist of four isoprene units and have the molecular formula C₂₀H₃₂. Diterpenes may be linear (acyclic) or contain rings. Examples of diterpenes include, but are not limited to, cembrene A, and taxadiene. A diterpene containing oxygen functionality or missing a methyl group is referred to herein as a diterpenoid. Examples of diterpenoids include, but are not limited to, cafestol, kahweol, retinol, retinal, and phytal.

As used herein, “sesterpene” refers to a class of terpenes that consist of five isoprene units and have the molecular formula C₂₅H₄₀. Sesterpenes may be linear (acyclic) or contain rings. A sesterpene containing oxygen functionality or missing a methyl group is referred to herein as a sesterpenoid.

As used herein, “triterpene” refers to a class of terpenes that consist of five isoprene units and have the molecular formula C₃₀H₄₈. Triterpenes may be linear (acyclic) or contain rings. A triterpene containing oxygen functionality or missing a methyl group is referred to herein as a triterpenoid.

As used herein, “tetraterpene” refers to a class of terpenes that consist of eight isoprene units and have the molecular formula C₄₀H₆₄. Tetraterpenes may be linear (acyclic) or contain rings. A tetraterpene containing oxygen functionality or missing a methyl group is referred to herein as a tetraterpenoid.

As used herein, “alkyl alcohol” is an alcohol having a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. In one aspect, the alkyl alcohol is a branched or unbranched C₁ to C₁₀ alcohol. In another aspect, alkyl alcohol is methanol, ethanol, propanol, isopropanol, butanol, pentanol, hexanol, heptanol, octanol, 2-ethyl hexanol, cetyl alcohol, or any combination thereof.

As used herein, “aryl alcohol” is an alcohol having any carbon-based aromatic group including, but not limited to, benzene, naphthalene, etc. The term “aromatic group” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. In one aspect, the heteroaryl group is imidazole. The aromatic group can be substituted or unsubstituted. The aromatic group can be substituted with one or more groups including, but not limited to, alkyl, alkynyl, alkenyl, aryl, halide, nitro, amino, ester, ketone, aldehyde, hydroxy, carboxylic acid, or alkoxy.

As used herein, “alkoxy alcohol” is an alcohol having the general formula R—O—R′—OH, where R and R′ are the same or different alkyl group as provided above.

As used herein, a plurality of items, structural elements, compositional elements, and/or materials may be presented in a common list for convenience. However, these lists should be construed as though each member of the list is individually identified as a separate and unique member. Thus, no individual member of any such list should be construed as a de facto equivalent of any other member of the same list based solely on its presentation in a common group, without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range was explicitly recited. As an example, a numerical range of “about 1” to “about 5” should be interpreted to include not only the explicitly recited values of about 1 to about 5, but also to include individual values and sub-ranges within the indicated range. Thus, included in this numerical range are individual values such as 2, 3, and 4, the sub-ranges such as from 1-3, from 2-4, from 3-5, from about 1-about 3, from 1 to about 3, from about 1 to 3, etc., as well as 1, 2, 3, 4, and 5, individually. The same principle applies to ranges reciting only one numerical value as a minimum or maximum. The ranges should be interpreted as including endpoints (e.g., when a range of “from about 1 to 3” is recited, the range includes both of the endpoints 1 and 3 as well as the values in between). Furthermore, such an interpretation should apply regardless of the breadth or range of the characters being described.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc. of these materials are disclosed, that while specific reference to each various individual combination and permutation of these compounds may not be explicitly disclosed, each is specifically contemplated and described herein. For example, if an alcohol is disclosed and discussed, and a number of different surfactants are discussed, each and every combination of alcohol and surfactant that is possible is specifically contemplated unless specifically indicated to the contrary. For example, if a class of alcohols A, B, and C are disclosed, as well as a class of surfactants D, E, and F, and an example combination of A+D is disclosed, then even if each is not individually recited, each is individually and collectively contemplated. Thus, in this example, each of the combinations A+E, A+F, B+D, B+E, B+F, C+D, C+E, and C+F is specifically contemplated and should be considered from disclosure of A, B, and C; D, E, and F; and the example combination A+D. Likewise, any subset or combination of these is also specifically contemplated and disclosed. Thus, for example, the sub-group of A+E, B+F, and C+E is specifically contemplated and should be considered from disclosure of A, B, and C; D, E, and F; and the example combination of A+D. This concept applies to all aspects of the disclosure including, but not limited to, steps in methods of making and using the disclosed compositions. Thus, if there are a variety of additional steps that can be performed with any specific embodiment or combination of embodiments of the disclosed methods, each such composition is specifically contemplated and should be considered disclosed.

The methods described herein are useful in reducing odor produced by a terpene. As used herein, the term “reduce” is defined as the ability to reduce the likelihood of detecting the odor produced by the terpene up to about 50%, up to about 60%, up to about 70%, up to about 80%, up to about 90%, up to about 95%, or up to about 99% when compared to not using the methods as described herein. As used herein, the term “reduce” is also defined as the ability to completely eliminate the likelihood of detecting the odor produced by the terpene when compared to not using the methods as described herein. The methods described herein are useful in reducing the odor produced by hemiterpenes, monoterpenes, sesquiterpenes, diterpenes, sesterpenes, triterpenes, tetraterpenes, or polyterpenes.

The methods described herein reduce the odor produced by a plurality of (i.e., two or more) of terpenes. It is understood that each terpene produces a distinct odor. The methods described herein reduce the odor produced collectively by the plurality of terpenes.

In one aspect, described herein is method for reducing the odor produced by a terpene, wherein the method comprises circulating air comprising the terpene through a first composition comprising an alcohol and a surfactant. In one aspect, the first composition is water based, where the first composition is composed of up about 70 vol %, up to about 75 vol %, up to about 80 vol %, up to about 85 vol %, up to about 90 vol %, up to about 95 vol %, up to about 98 vol %, or up to about 99 vol % water. In another aspect, the amount of water in the first composition is about 70 vol %, about 75 vol %, about 80 vol %, about 85 vol %, about 90 vol %, about 95 vol %, about 96 vol %, about 97 vol %, about 98 vol %, or about 99 vol % water, where any value can be a lower and upper endpoint of a range (e.g., about 80 vol % to about 90 vol %, about 96 vol % to about 98 vol %, etc.).

In one aspect, the alcohol in the first composition is an alkyl alcohol, aryl alcohol, alkoxy alcohol, or any combination thereof. The selection of the alcohol can vary depending upon the terpene that is present in the air. In one aspect, the alcohol is selected based on the solubility of the terpene in the compositions described herein, wherein the alcohol helps keep the terpene in the compositions. In another aspect, the alcohol in the first composition is a branched or linear C₁ to C₁₀ alcohol. In one aspect, the alcohol in the first composition is methanol, ethanol, propanol, isopropanol, butanol, pentanol, hexanol, heptanol, octanol, 2-ethyl hexanol, cetyl alcohol, dodecyl alcohol, or any combination thereof. In one aspect, the amount of alcohol in the first composition is from about 0.5 vol % to about 10 vol %, or about 0.5 vol %, about 1 vol %, about 1.5 vol %, about 2 vol %, about 2.5 vol %, about 3 vol %, about 3.5 vol %, about 4 vol %, about 4.5 vol %, about 5 vol %, about 5.5 vol %, about 6 vol %, about 6.5 vol %, about 7 vol %, about 7.5 vol %, about 8 vol %, about 8.5 vol %, about 9 vol %, about 9.5 vol %, or about 10 vol %, where any value can be a lower and upper endpoint of a range (e.g., about 1 vol % to about 5 vol %, about 2 vol % to about 4 vol %, etc.).

In certain aspects, two alcohols can be present in the first composition. In one aspect, the alcohol comprises isopropanol and 2-ethyl hexanol. In another aspect, the volume ratio of isopropanol to 2-ethyl hexanol in the first composition is from about 1:0.5 to about 1:4, or is about 1:0.5, about 1:1, about 1:1.5, about 1:2, about 1:2.5, about 1:3, about 1:3.5, or about 1:4, where any ratio can be a lower and upper endpoint of a range (e.g., about 1:1 to about 1:3, about 1.5 to about 1.35, etc.).

The first composition includes a surfactant. In one aspect, the surfactant is a nonionic surfactant, an anionic surfactant, an amphoteric surfactant, a zwitterionic surfactant, or any combination thereof.

In one aspect, the nonionic surfactant useful herein can include alkoxylated fatty acid esters, alkoxylated fatty alcohols, alkyl glucosides, alkyl polyglucosides, amine oxides, cocoamine oxide, glyceryl monohydroxystearate, glyceryl stearate, hydroxy stearic acid, lauramine oxide, laureth-2, polyhydroxy fatty acid amides, polyoxyalkylene stearates, propylene glycol stearate, sorbitan monostearate, sucrose cocoate, sucrose esters, sucrose laurate, steareth-2, polyethylene glycol, polypropylene glycol, or any combination thereof. Several commercial forms of nonionic surfactants with the trade name Triton, are also available, and will be very useful herein. Examples of such surfactants include, but are not limited to, Triton® X-180, Triton® X-193, and Triton® X-405 available from Dow Chemical; and Empilan® MAA and Emplian® NP-S from Albright and Wilson, Ltd. In one aspect, the nonionic surfactant can be an alkoxylated fatty alcohol.

In other aspects, the nonionic surfactant can include alkoxylated fatty alcohols. The nonionic surfactant may be selected from ethoxylated alcohols and ethoxylated alkyl phenols of the formula R(OC₂H₄)ₙOH, wherein R is selected from the group consisting of aliphatic hydrocarbon radicals containing from about 8 to about 15 carbon atoms and alkyl phenyl radicals in which the alkyl groups contain from about 8 to about 12 carbon atoms, and the average value of n is from about 5 to about 15.

Other non-limiting examples of nonionic surfactants useful herein include: C₈-C₁₈ alkyl ethoxylates, such as, NEODOL® nonionic surfactants from Shell; C₆-C₁₂ alkyl phenol alkoxylates where the alkoxylate units may be ethyleneoxy units, propyleneoxy units, or a mixture thereof; C₁₂-C₁₈ alcohol and C₆-C₁₂ alkyl phenol condensates with ethylene oxide/propylene oxide block polymers such as Pluronic® from BASF; C₁₄-C₂₂ mid-chain branched alcohols, BA; C₁₄-C₂₂ mid-chain branched alkyl alkoxylates, BAEₓ, wherein x is from 1 to 30; alkylpolysaccharides; specifically alkylpolyglycosides; polyhydroxy fatty acid amides; and ether capped poly(oxyalkylated) alcohol surfactants.

In one aspect, the anionic surfactant useful herein can include alcohol phosphates, alkyl alkoxy carboxylates, alkyl aryl sulfates, alkyl aryl sulfonates, alkyl carboxylates, alkyl ether carboxylates, alkyl ether sulfates, alkyl ether sulfonates, alkyl phosphates, alkyl polyethoxy carboxylates, alkyl polyglucosides, alkyl polyglucoside sulfates, alkyl polyglucoside sulfonates, alkyl succinamates, alkyl sulfates, alkyl sulfonates, aryl sulfates, aryl sulfonates, fatty taurides, isethionates, N-acyl taurates, nonoxynol phosphates, octoxynol phosphates, sarcosinates, sulfated fatty acid esters, taurates, and mixtures thereof. In one aspect, the anionic surfactant is an alkyl benzene sulfonate such as, for example, dodecyl benzene sulfonic acid (DDBSA).

In another aspect, the anionic surfactant is an alkoxylated and/or non-alkoxylated alkyl sulfate materials, and/or sulfonic detersive surfactants, e.g., alkyl benzene sulfonates. Suitable anionic surfactants may be derived from renewable resources, waste, petroleum, or mixtures thereof. Suitable anionic surfactants may be linear, partially branched, branched, or mixtures thereof.

In one aspect, the anionic surfactant is an alkoxylated alkyl sulfate materials comprise ethoxylated alkyl sulfate surfactants, also known as alkyl ether sulfates or alkyl polyethoxylate sulfates. Examples of ethoxylated alkyl sulfates include water-soluble salts, particularly the alkali metal, ammonium and alkylolammonium salts, of organic sulfuric reaction products having in their molecular structure an alkyl group containing from about 8 to about 30 carbon atoms and a sulfonic acid and its salts. In some examples, the alkyl group contains from about 15 carbon atoms to about 30 carbon atoms. In other examples, the alkyl ether sulfate surfactant may be a mixture of alkyl ether sulfates, said mixture having an average (arithmetic mean) carbon chain length within the range of about 12 to 30 carbon atoms, and in some examples an average carbon chain length of about 12 to 15 carbon atoms, and an average (arithmetic mean) degree of ethoxylation of from about 1 mol to 4 mols of ethylene oxide, and in some examples an average (arithmetic mean) degree of ethoxylation of 1.8 mols of ethylene oxide. In further examples, the alkyl ether sulfate surfactant may have a carbon chain length between about 10 carbon atoms to about 18 carbon atoms, and a degree of ethoxylation of from about 1 to about 6 mols of ethylene oxide. In yet further examples, the alkyl ether sulfate surfactant may contain a peaked ethoxylate distribution.

In another aspect, the anionic surfactant is a non-alkoxylated alkyl sulfates. Examples of non-alkoxylated, e.g., non-ethoxylated, alkyl sulfate surfactants include those produced by the sulfation of higher C₈-C₂₀ fatty alcohols. In some examples, primary alkyl sulfate surfactants have the general formula: ROSO3⁻M⁺, wherein R is typically a linear C₈-C₂₀ hydrocarbyl group, which may be straight chain or branched chain, and M is a water-solubilizing cation. In some examples, R is a C₁₀-C₁₈ alkyl, and M is an alkali metal. In other examples, R is a C₁₂/C₁₄ alkyl and M is sodium, such as those derived from natural alcohols.

Other useful anionic surfactants can include the alkali metal salts of alkyl benzene sulfonates, in which the alkyl group contains from about 9 to about 15 carbon atoms, in straight chain (linear) or branched chain configuration. In some examples, the alkyl group is linear. Such linear alkylbenzene sulfonates are known as “LAS.” In other examples, the linear alkylbenzene sulfonate may have an average number of carbon atoms in the alkyl group of from about 11 to 14. In a specific example, the linear straight chain alkyl benzene sulfonates may have an average number of carbon atoms in the alkyl group of about 11.8 carbon atoms, which may be abbreviated as C11.8 LAS.

In another aspect, the anionic surfactant can include a 2-alkyl branched primary alkyl sulfate having 100% branching at the C2 position (C1 is the carbon atom covalently attached to the alkoxylated sulfate moiety). 2-alkyl branched alkyl sulfates and 2-alkyl branched alkyl alkoxy sulfates are generally derived from 2-alkyl branched alcohols (as hydrophobes). 2-alkyl branched alcohols, e.g., 2-alkyl-1-alkanols or 2-alkyl primary alcohols, which are derived from the oxo process, are commercially available from Sasol, e.g., LIAL®, ISALCHEM®, which is prepared from LIAL® alcohols by a fractionation process. C₁₄/C₁₅ branched primary alkyl sulfate are also commercially available, e.g., namely LIAL® 145 sulfate.

In other aspects, the anionic surfactant can be a mid-chain branched anionic surfactant, e.g., a mid-chain branched anionic detersive surfactant, such as, a mid-chain branched alkyl sulphate and/or a mid-chain branched alkyl benzene sulphonate. In other aspects, the anionic surfactant can include methyl ester sulfonates, paraffin sulfonates, α-olefin sulfonates, and internal olefin sulfonates.

In one aspect, the surfactant is a cationic surfactant. Non-limiting examples of cationic surfactants include: the quaternary ammonium surfactants, which can have up to 26 carbon atoms include: alkoxylate quaternary ammonium (AQA) surfactants; dimethyl hydroxyethyl quaternary ammonium; dimethyl hydroxyethyl lauryl ammonium chloride; polyamine cationic surfactants; cationic ester surfactants; and amino surfactants, e.g., amido propyldimethyl amine (APA)

In one aspect, the cationic surfactant can include alkyl pyridinium compounds, alkyl quaternary ammonium compounds, alkyl quaternary phosphonium compounds, alkyl ternary sulphonium compounds, and mixtures thereof. In another aspect, the cationic surfactant can be a quaternary ammonium compounds having the general formula (R)(R₁)(R₂)(R₃)N⁺X⁻, wherein, R is a linear or branched, substituted or unsubstituted C₆₋₁₈ alkyl or alkenyl moiety, R₁ and R₂ are independently selected from methyl or ethyl moieties, R₃ is a hydroxyl, hydroxymethyl or a hydroxyethyl moiety, X is an anion which provides charge neutrality, suitable anions include: halides, for example chloride; sulphate; and sulphonate. Suitable cationic surfactants include mono-C₆₋₁₈ alkyl mono-hydroxyethyl di-methyl quaternary ammonium chlorides. Highly suitable cationic detersive surfactants are mono-C₈₋₁₀ alkyl mono-hydroxyethyl di-methyl quaternary ammonium chloride, mono-C₁₀₋₁₂ alkyl mono-hydroxyethyl di-methyl quaternary ammonium chloride and mono-C₁₀ alkyl mono-hydroxyethyl di-methyl quaternary ammonium chloride.

In one aspect, the surfactant is a zwitterionic surfactant. Suitable zwitterionic surfactants include: derivatives of secondary and tertiary amines, derivatives of heterocyclic secondary and tertiary amines, or derivatives of quaternary ammonium, quaternary phosphonium or tertiary sulfonium compounds. Suitable examples of zwitterionic surfactants include betaines, including alkyl dimethyl betaine and cocodimethyl amidopropyl betaine, C₈ to C₁₈ (for example from C₁₂ to C₁₈) amine oxides, and sulfo and hydroxy betaines, such as N-alkyl-N,N-dimethylammino-1-propane sulfonate where the alkyl group can be C₈ to C₁₈.

In one aspect, the surfactant is an amphoteric surfactant. Suitable amphoteric surfactants include aliphatic derivatives of secondary or tertiary amines, or aliphatic derivatives of heterocyclic secondary and tertiary amines in which the aliphatic radical may be straight or branched-chain and where one of the aliphatic substituents contains at least about 8 carbon atoms, or from about 8 to about 18 carbon atoms, and at least one of the aliphatic substituents contains an anionic water-solubilizing group, e.g. carboxy, sulfonate, sulfate. Suitable amphoteric surfactants also include sarcosinates, glycinates, taurinates, and mixtures thereof.

In one aspect, the surfactant can be part of or included in a detergent. In one aspect, the detergent is DAWN® dishwashing liquid manufactured by Procter & Gamble, which is a mixture of sodium laureth sulfate, denatured alcohol, phenoxyethanol, sodium chloride, PPG-26 (propylene glycol), PEG-8, propylheptyl ether, methylisothiazolinone, fragrance, yellow 5, blue 1.

In one aspect, the amount of surfactant in the first composition is from about 0.1 vol % to about 5 vol %, or about 0.1 vol %, about 0.5 vol %, about 1 vol %, about 1.5 vol %, about 2 vol %, about 2.5 vol %, about 3 vol %, about 3.5 vol %, about 4 vol %, about 4.5 vol %, or about 5 vol %, where any value can be a lower and upper endpoint of a range (e.g., about 0.1 vol % to about 3 vol %, about 0.5 vol % to about 2 vol %, etc.). In another aspect, the surfactant is DAWN® dishwashing liquid in the amount of about 0.1 vol % to about 5 vol %, or about 0.1 vol %, about 0.5 vol %, about 1 vol %, about 1.5 vol %, about 2 vol %, about 2.5 vol %, about 3 vol %, about 3.5 vol %, about 4 vol %, about 4.5 vol %, or about 5 vol %, where any value can be a lower and upper endpoint of a range (e.g., about 0.1 vol % to about 3 vol %, about 0.5 vol % to about 2 vol %, etc.).

In one aspect, described herein is method for reducing the odor produced by a terpene, wherein the method comprises circulating air comprising the terpene through a second composition comprising an oxidizing agent. In one aspect, the second composition is water based, where the second composition is composed of up to about 70 vol %, up to about 75 vol %, up to about 80 vol %, up to about 85 vol %, up to about 90 vol %, up to about 95 vol %, up to about 98 vol %, or up to about 99 vol % water. In another aspect, the amount of water in the second composition is about 70 vol %, about 75 vol %, about 80 vol %, about 85 vol %, about 90 vol %, about 95 vol %, about 96 vol %, about 97 vol %, about 98 vol %, or about 99 vol % water, where any value can be a lower and upper endpoint of a range (e.g., about 80 vol % to about 90 vol %, about 96 vol % to about 98 vol %, etc.).

Examples of oxidizing agents useful herein include, but are not limited to, a peroxide (e.g., hydrogen peroxide, benzoyl peroxide, t-butyl hydrogen peroxide), a monosulfate (e.g., sodium or potassium peroxomonosulfate), a persulfate (e.g., sodium potassium or ammonium persulfate), a perborates, a carbonate peroxyhydrate, a phosphate peroxyhydrate, or any combination thereof.

In another aspect, the oxidizing agent can be chlorine-containing compounds such as an alkali metal hypochlorite, an alkali metal dichloroisocyanurate, a chlorinated trisodium phosphate, or any combination thereof.

In one aspect, the amount of oxidizing agent in the second composition is one or more oxidizing agents in the total amount of up to about 10 dry wt %, or about 0.1 dry wt %, about 0.5 dry wt %, about 1 dry wt %, about 1.5 dry wt %, about 2 dry wt %, about 2.5 dry wt %, about 3 dry wt %, about 3.5 dry wt %, about 4 dry wt %, about 4.5 dry wt %, about 5 dry wt %, about 5.5 dry wt %, about 6 dry wt %, about 6.5 dry wt %, about 7 dry wt %, about 7.5 dry wt %, about 8 dry wt %, about 8.5 dry wt %, about 9 dry wt %, about 9.5 dry wt %, or about 10 dry wt %, where any value can be a lower and upper endpoint of a range (e.g., about 1 dry wt % to about 8 dry wt %, about 2 dry wt % to about 4 dry wt %, etc.).

In certain aspects, the second composition further includes a reducing agent. In one aspect, the reducing agent is a hypophosphorous acid or a salt thereof, a metabisulfite salt, sulfite salt, a bisulfite salt, or any combination thereof. In one aspect, the reducing agent is an alkali metal sulfite, alkali metal bisulfite, sodium formaldehyde sulfoxylate (NaHSO₂HCHO), an alkali salt of an organic sulfinic acid derivative, ascorbic acid, glucose, or a catalytic system such as, for example, ammonium, sodium, or potassium persulfate/sodium metabisulfite, diisopropylbenzene hydroperoxide/sodium formaldehyde sulfoxylate, ferrous sulfate/dextrose/sodium pyrophosphate.

In one aspect, the amount of reducing agent in the second composition is up to about 5 dry wt %, or about 0.1 dry wt %, about 0.5 dry wt %, about 1 dry wt %, about 1.5 dry wt %, about 2 dry wt %, about 2.5 dry wt %, about 3 dry wt %, about 3.5 dry wt %, about 4 dry wt %, about 4.5 dry wt %, or about 5 dry wt %, where any value can be a lower and upper endpoint of a range (e.g., about 0.5 dry wt % to about 4 dry wt %, about 1 dry wt % to about 3 dry wt %, etc.).

In one aspect, described herein is method for reducing the odor produced by a terpene, wherein the method comprises circulating air comprising the terpene through a third composition comprising an acid. In one aspect, the acid is a carboxylic acid such as, for example, those represented by the formula RCO₂H, where R is an alkyl or aryl group as defined herein. In another aspect, the acid is acetic acid, propionic acid, citric acid, butyric acid, pentanoic acid, hexanoic acid, or any combination thereof in water. In one aspect, the concentration of the acid is from about 1 vol % to about 10 vol %, or about 1 vol %, about 1.5 vol %, about 2 vol %, about 2.5 vol %, about 3 vol %, about 3.5 vol %, about 4 vol %, about 4.5 vol %, about 5 vol %, about 5.5 vol %, about 6 vol %, about 6.5 vol %, about 7 vol %, about 7.5 vol %, about 8 vol %, about 8.5 vol %, about 9 vol %, about 9.5 vol %, or about 10 vol %, where any value can be a lower and upper endpoint of a range (e.g., about 2 vol % to about 5 vol %, etc.).

In certain aspects, any of the compositions described herein for reducing odors produced from terpenes can include a defoamer. As used herein, a “defoamer” is a chemical added to the compositions disclosed herein to reduce and/or hinder foam formation in the composition. In one aspect, the defoamer can be petroleum hydrocarbons, an alcohol as described herein, sulfonated oils, organic phosphates, silicone fluids, dimethylpolysiloxanes, polyalkoxylated polyethers, mineral oils, vegetable oils, polyethylene glycol dioleate and propylene glycol monooleate, or a combination thereof. The defoamer may further comprise a wax, hydrophobic silica, or a combination thereof. In a further aspect, the wax can be ethylene bis-stearamide, paraffinic waxes, ester waxes, and fatty alcohol waxes. Various commercial defoamers are also contemplated; these include, but are not limited to: Foamkill® 600 Series from Crucible Chemical, Foammizer® M-55 from C. P. Hall, and Nalco® 5770 and 5772 from Nalco Chemical. In one aspect, Foamaster® 111 from Henkel Corporation can be used as the defoamer. Certain bactericides such as Amerstate® 251 from Drew Chemical may also reduce foaming. The above list of compounds is not meant to be limiting and various other defoamers known in the art can be used herein.

In one aspect, the defoamer is a polyoxyethylene-polyoxypropylene copolymer. An example of a polyoxyethylene-polyoxypropylene copolymer useful herein as a defoamer includes Belite® M8 manufactured by BWA, which is composed of the copolymer as well as polyethylene glycol dioleate, propylene glycol monooleate, and water.

In one aspect, the defoamer can be from about 0.1 vol % to about 1.0 vol %, or about 0.1 vol %, about 0.2 vol %, about 0.3 vol %, about 0.4 vol %, about 0.5 vol %, about 0.6 vol %, about 0.7 vol %, about 0.8 vol %, about 0.9 vol %, or about 1.0 vol % of the compositions described herein, where any value can be a lower and upper endpoint of a range (e.g., about 0.1 vol % to about 0.8 vol %, about 0.2 vol % to about 0.6 vol %, etc.).

In certain aspects, the compositions described herein can include pH adjusters. In one aspect, the adjuster can be an acidic pH material such as, for example, an alpha or beta hydroxycarboxylic acid. The alpha or beta hydroxycarboxylic acid may be selected from the group comprising salicylic acid, glycolic acid, mandelic acid, lactic acid, tartaric acid, malic acid, citric acid, isocitric acid, alpha hydroxybutanoic acid, alpha hydroxyhexanoic acid, alpha hydroxyoctanoic acid, alpha hydroxynonanoic acid, alpha hydroxydecanoic acid, alpha hydroxyundecanoic acid, alpha hydroxydodecanoic acid, alpha hydroxytetradecanoic acid, alpha hydrocyhexadecanoic acid, alpha hydroxyoctadecanoic acid, alpha hydroxyoctaeicosanoic acid, dicarboxylic alpha hydroxy acids, 2-hydroxy propanedioic acid, 2-hydroxy hexanedioic acid, 2-hydroxy octanedioic acid, 2-hydroxy decanedioic acid, 2-hydroxy dodecanedioic acid, 2-hydroxy myristicdioic acid, 2-hydroxy palmiticdioic acid, tricarboxylic alpha hydroxy acid, their ester or salt derivatives, or combinations thereof. The above list of compounds is not meant to be limiting and various other stabilizers known in the art can be used herein.

In another aspect, the pH adjuster can be a basic pH material such as, for example, caustic soda, caustic potash, ammoniated tallow, dimethyl amine, ammonia, dimethyl amino ethanol, urea, diethanolamine, triethanolamine, or morpholine. In another aspect, the pH adjuster is sodium carbonate or sodium bicarbonate.

The compositions described herein can be produced by admixing two or more components together. In one aspect, with respect to the first composition, the alcohol and surfactant can be admixed with one another and stored indefinitely until ready for use. In this aspect, the alcohol and surfactant can subsequently added to water at a desired concentration. In other aspects, the components can be added sequentially to water (e.g., alcohol then surfactant, surfactant then alcohol, etc.).

The air composed the terpenes can be circulated through the compositions described herein by blowers, fans, air-handlers and other related devices. In one aspect, air comprising the terpenes is fed through a pipe connected to a dip tube inside a tank, where the tank holds the compositions described herein. The tank can also include an outlet where the air treated by the composition is released.

In certain aspects, prior to circulating the air comprising the terpene through the compositions described herein, the air is heated. Not wishing to be bound by theory, heating the air comprising the terpenes can autoxidize and/or autopolymerize the terpenes. In one aspect, prior to circulating the air comprising the terpene through the compositions described herein, the air is heated at a temperature of from about 25° C. to about 85° C. In another aspect, the air is heated at a temperature of from about 25° C., about 30° C., about 35° C., about 40° C., about 45° C., about 50° C., about 55° C., about 60° C., about 65° C., about 70, about 75° C., about 80, or about 85° C., where any value can be a lower and upper endpoint of a range (e.g., about 25° C. to about 60° C., about 30° C. to about 50° C., etc.). The air can be heated using equipment known in the art such as, for example, a catalytic converter, a furnace, or a thermal unit.

In certain aspects, after the air comprising the terpenes has been circulated through the composition described herein, the air can be further treated to ensure the most if not all of the terpenes have been removed from the circulating air. In one aspect, after the air comprising the terpenes has been circulated through the composition, the air can be subsequently circulated through water or any of the compositions described herein (e.g., first or second composition). In one aspect, air comprising the terpenes is fed through a pipe connected to a first tank, where the first tank holds the compositions described herein. The first tank includes an outlet and pipe connected to a second tank containing water, where the air treated by the composition exits the first tank through the outlet into the second tank. The second tank also includes an outlet where the air that is treated by the water is released.

In another aspect, after the air comprising the terpenes has been circulated through the composition, the air can be subsequently circulated through activated charcoal. In one aspect, air comprising the terpenes is fed through a pipe connected to a first tank, where the first tank holds the compositions described herein. The first tank includes an outlet and pipe connected to a container housing the activated charcoal, where the air treated by the composition exits the first tank through the outlet into the container. The container with the activated charcoal also includes an outlet where the air that is filtered through the activated charcoal is released.

In another aspect, after the air comprising the terpenes has been circulated through the composition, the air can be subsequently cooled. In one aspect, air comprising the terpenes is fed through a pipe connected to a first tank, where the first tank holds the compositions described herein. The first tank includes an outlet and pipe connected to a cooling device (e.g., a condenser). In one aspect, the air is cooled at a temperature of from about 20° C. to about 50° C. In another aspect, the air is cooled at a temperature of from about 20° C., about 25° C., about 30° C., about 35° C., about 40° C., about 45° C., about 50° C., where any value can be a lower and upper endpoint of a range (e.g., about 20° C. to about 40° C., about 25° C. to about 30° C., etc.).

In one aspect, after the air comprising the terpenes has been circulated through the composition, the air can be subsequently circulated through water followed by activated charcoal. In another aspect, after the air comprising the terpenes has been circulated through the composition, the air can be subsequently circulated through activated charcoal followed by water.

In one aspect, after the air comprising the terpenes has been circulated through the composition, the air can be subsequently circulated through water followed by cooling the air. In another aspect, after the air comprising the terpenes has been circulated through the composition, the air can be optionally cooled then circulated through water.

In one aspect, after the air comprising the terpenes has been circulated through the composition, the air can be subsequently circulated through activated charcoal followed by optional cooling of the air. In another aspect, after the air comprising the terpenes has been circulated through the composition, the air can be optionally be cooled then circulated through activated charcoal.

In one aspect, after the air comprising the terpenes has been circulated through the composition, the air can be subsequently circulated through activated charcoal and water (i.e., charcoal then water or water then charcoal) followed by optional cooling of the air. In another aspect, after the air comprising the terpenes has been circulated through the composition, the air can be optionally be cooled then circulated through activated charcoal and water (i.e., charcoal then water or water then charcoal). In another aspect, after the air comprising the terpenes has been circulated through the composition, the air can be subsequently circulated through activated charcoal, optionally cooled, and then circulated through water. In another aspect, after the air comprising the terpenes has been circulated through the composition, the air can be subsequently circulated through water, optionally cooled, and then circulated through activated charcoal.

In certain aspects, the terpenes are present in an enclosed structure where it is desirable to reduce the odor produced by the terpenes. In one aspect, the enclosed structure is a greenhouse where plants are cultivated that produce terpenes. In another aspect, the enclosed structure is a storage room or container, a transport container (e.g., a rail car) that contains terpenes or products that produce terpenes (e.g., plants). In another aspect, the enclosed structure is processing room where terpenes are handled.

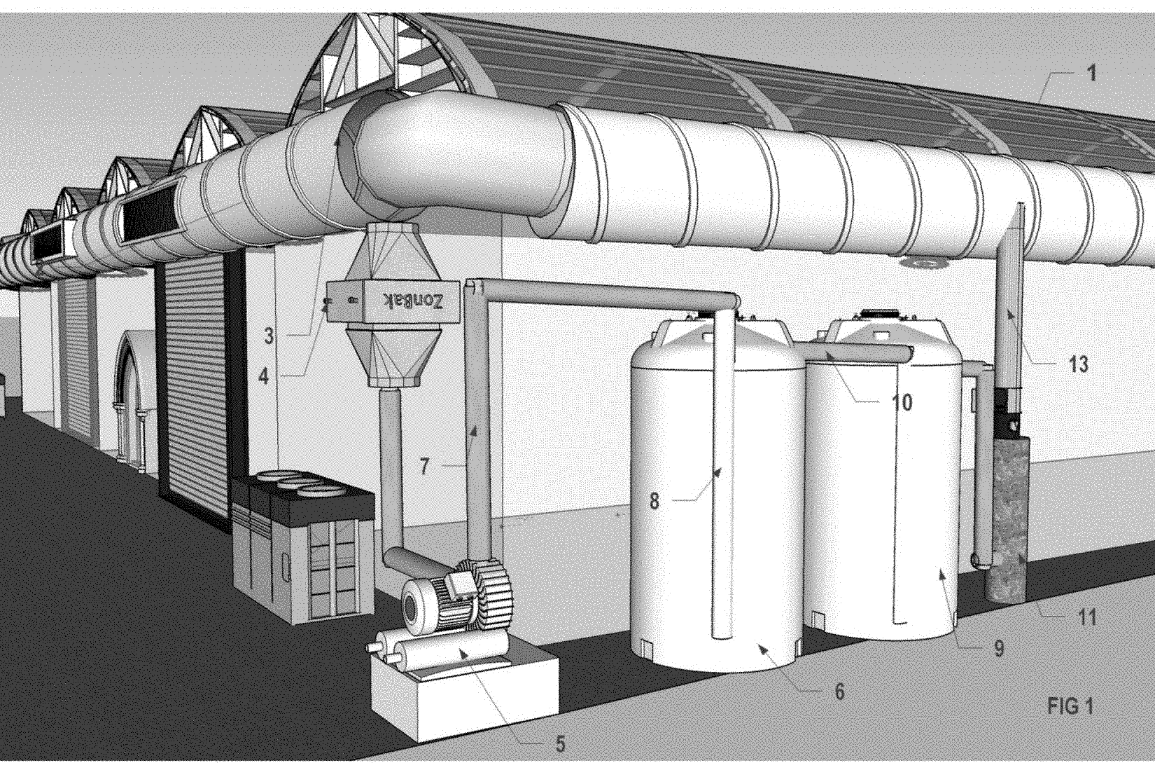

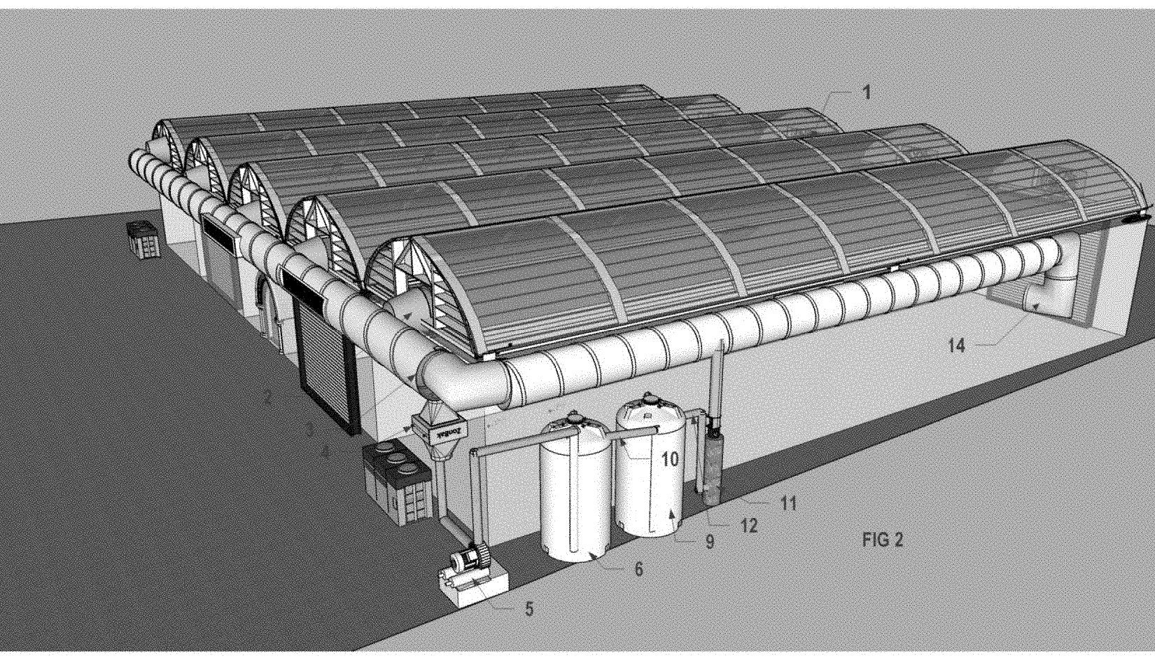

In certain aspects, after the air comprising the terpenes has been circulated through the compositions described herein and optionally treated (e.g., circulated through water and/or activated charcoal, cooled, etc.), the air can be re-circulated into an enclosed structure. FIGS. 1 and 2 provide a non-limiting example of this aspect.

Referring to FIGS. 1 and 2, the greenhouse 1 is fitted with a fan 2 and duct 3. Air from the greenhouse 1 is circulated through duct 3 through a heating unit 4 (optional). The blower 5 pulls air from the heating unit 4 into first tank 6 through pipe 7. The first tank 6 contains the compositions described herein. The first tank includes a second pipe 8, where the second pipe 8 is within the first tank 6. The second pipe 8 is intended to be immersed in the compositions described herein. The first and second pipes are configured to remove the contents of the tanks (e.g., spent composition, water, etc.). The size of the tanks can vary depending upon size of the greenhouse 1 and the pressure of the air that is circulated through the tanks.

The first tank 6 is connected to a second tank 9 via pipe 10. The second tank is optional, and can include water or a composition as described herein. As depicted in FIGS. 1 and 2, the second tank is connected to a container unit 11 filled with activated charcoal via pipe 12. The container unit 11 is connected to a clean return 13, where air is re-circulated back to duct 3 and into the greenhouse 1 via duct outlet 14.

In one aspect, described herein is method for reducing the odor produced by a terpene, wherein the method comprises (1) heating the air that comprises the terpene and (2) circulating the air through activated charcoal. The air can be heated using any of the heating devices described herein (e.g., catalytic converter, furnace, thermal unit). In one aspect, the air is heated at a temperature of from about 25° C. to about 85° C. In another aspect, the air is heated at a temperature of from about 25° C., about 30° C., about 35° C., about 40° C., about 45° C., about 50° C., about 55° C., about 60° C., about 65° C., about 70, about 75° C., about 80, or about 85° C., where any value can be a lower and upper endpoint of a range (e.g., about 25° C. to about 60° C., about 30° C. to about 50° C., etc.).

The methods described herein can reduce the odor produced from terpenes in a variety of different settings. In one aspect, the methods described herein are useful in reducing odors where terpenes are handled, processed, synthesized, purified, or chemically modified.

In another aspect, the source of the terpenes is a plant that naturally produces terpenes. In one aspect, the plant is a conifer. In another aspect, the terpene is produced from a plant from the genus Cannabis. In another aspect, the terpene is produced from the species Cannabis sativa, Cannabis indica, or Cannabis ruderalis. In another aspect, the terpene is produced from the flowers, stems, or leaves of Cannabis sativa. In another aspect, the source of the terpenes is from hemp. Not wishing to be bound by theory, the terpenes produced by Cannabis sativa are predominantly monoterpenes and minor component of sesquiterpenes.

The following listing of exemplary aspects supports and is supported by the disclosure provided herein.

Aspect 1. A method for reducing the odor produced by a terpene, the method comprising circulating air comprising the terpene through a composition comprising an alcohol and a surfactant.

Aspect 2. The method according to Aspect 1, wherein the solution further comprises water.

Aspect 3. The method according to Aspect 1, wherein the alcohol is an alkyl alcohol, aryl alcohol, alkoxy alcohol, or any combination thereof.

Aspect 4. The method according to Aspect 1, wherein the alcohol is a branched or linear C₁ to C₁₀ alcohol.

Aspect 5. The method according to Aspect 1, wherein the alcohol is methanol, ethanol, propanol, isopropanol, butanol, pentanol, hexanol, heptanol, octanol, 2-ethyl hexanol, cetyl alcohol, dodecyl alcohol, or any combination thereof.

Aspect 6. The method according to Aspect 1, wherein the surfactant comprises a nonionic surfactant, an anionic surfactant, a cationic surfactant, an amphoteric surfactant, a zwitterionic surfactant, or any combination thereof.

Aspect 7. The method according to Aspect 1, wherein the surfactant comprises a nonionic surfactant, and the nonionic surfactant comprises an alkoxylated fatty acid ester, an alkoxylated fatty alcohol, an alkyl glucoside, an alkyl polyglucoside, an amine oxide, cocoamine oxide, glyceryl monohydroxystearate, glyceryl stearate, hydroxy stearic acid, lauramine oxide, a polyhydroxy fatty acid amide, a polyoxyalkylene stearate, propylene glycol stearate, sorbitan monostearate, sucrose cocoate, a sucrose ester, sucrose laurate, steareth-2, polyethylene glycol, polypropylene glycol, or any combination thereof.

Aspect 8. The method according to Aspect 1, wherein the surfactant comprises an anionic surfactant, and the anionic surfactant comprises an alcohol phosphate, an alkyl alkoxy carboxylate, an alkyl aryl sulfate, an alkyl aryl sulfonate, an alkyl carboxylate, an alkyl ether carboxylate, an alkyl ether sulfate, an alkyl ether sulfonate, an alkyl phosphate, an alkyl polyethoxy carboxylate, an alkyl polyglucoside, an alkyl polyglucoside sulfate, an alkyl polyglucoside sulfonate, an alkyl succinamate, an alkyl sulfate, an alkyl sulfonate, an aryl sulfate, an aryl sulfonate, a fatty tauride, an isethionate, an N-acyl taurate, a nonoxynol phosphate, an octoxynol phosphate, a sarcosinate, a sulfated fatty acid ester, a taurate, an alkyl benzene sulfonate, or any combination thereof.

Aspect 9. The method according to Aspect 1, wherein the composition further comprises a defoamer.

Aspect 10. The method according to any one of Aspect 1-Aspect 9, wherein the alcohol comprises isopropanol and 2-ethyl hexanol.

Aspect 11. The method according to Aspect 10, wherein volume ratio of isopropanol to 2-ethyl hexanol is from about 1:0.5 to about 1:4.

Aspect 12. The method according to any one of Aspect 1-Aspect 11, wherein the surfactant comprises a detergent.

Aspect 13. The method according to any one of Aspect 1-Aspect 11, wherein the surfactant comprises sodium lauryl sulfate.

Aspect 14. A method for reducing the odor produced by a terpene, the method comprising circulating air comprising the terpene through a composition comprising an oxidizing agent.

Aspect 15. The method according to Aspect 14, wherein the solution further comprises water.

Aspect 16. The method according to Aspect 14, wherein the oxidizing agent comprises hydrogen peroxide, benzoyl peroxide, t-butyl hydrogen peroxide, di-t-butyl peroxide, t-butyl peroxy benzoate, cumyl hydro peroxide, di-cumyl peroxide, ketone peroxides, a monosulfate, a persulfate, a perborate, a carbonate peroxyhydrate, a phosphate peroxyhydrate, or any combination thereof.

Aspect 17. The method according to Aspect 14, wherein the oxidizing agent comprises an alkali metal hypochlorite, an alkali metal dichloroisocyanurate, a chlorinated trisodium phosphate, or any combination thereof.

Aspect 18. The method according to any one of Aspect 14-Aspect 17, wherein the oxidizing agent comprises hydrogen peroxide and sodium persulfate.

Aspect 19. The method according to any one of Aspect 14-Aspect 17, wherein the oxidizing agent is up to about 10 dry wt % of the composition.

Aspect 20. The method according to any one of Aspect 14-Aspect 17, wherein the oxidizing agent is from about 0.5 dry wt % to about 3 dry wt % of the composition.

Aspect 21. The method according to any one of Aspect 14-Aspect 17, wherein the oxidizing agent is two or more agents in the total amount of from about 1 dry wt % to about 5 dry wt % of the composition.

Aspect 22. The method according to any one of Aspect 14-Aspect 17, wherein the oxidizing agent is two or more agents in the total amount of from about 1 dry wt % to about 3 dry wt % of the composition.

Aspect 23. The method according to any one of Aspect 14-Aspect 22, wherein the composition further comprises a reducing agent.

Aspect 24. The method according to Aspect 23, wherein the reducing agent comprises hypophosphorous acid or a salt thereof, a metabisulfite salt, sulfite salt, a bisulfite salt, or any combination thereof.

Aspect 25. The method according to Aspect 23 or Aspect 24, wherein the reducing agent is from about 0.5 dry wt % to about 5 dry wt % of the composition.

Aspect 26. A method for reducing the odor produced by a terpene, the method comprising circulating air comprising the terpene through a composition comprising an acid.

Aspect 27. The method according to Aspect 26, wherein the acid comprises a carboxylic acid.

Aspect 28. The method according to Aspect 26, wherein the acid comprises acetic acid, propionic acid, citric acid, butyric acid, pentanoic acid, hexanoic acid, or any combination thereof in water.

Aspect 29. The method according to any one of Aspect 26-Aspect 28, wherein the concentration of the acid is from about 1 vol % to about 10 vol %.

Aspect 30. The method according to any one of Aspect 1-Aspect 29, wherein the composition further comprises a defoamer comprising a petroleum hydrocarbon, an alcohol, a sulfonated oil, an organic phosphate, a silicone fluid, a dimethylpolysiloxane, a polyalkoxylated polyether, a mineral oil, a vegetable oil, a wax, a hydrophobic silica, a polyoxyethylene-polyoxypropylene copolymer, polyethylene glycol dioleate and propylene glycol monooleate, or any combination thereof.

Aspect 31. The method according to any one of Aspect 1-Aspect 30, wherein prior to circulating the air comprising the terpene through the composition, heating the air at a temperature of from about 25° C. to about 85° C.

Aspect 32. The method according to Aspect 31, wherein the air is warmed by a catalytic converter, a furnace, or a thermal unit.

Aspect 33. The method according to any one of Aspect 1-Aspect 32, wherein after the air has been circulated through the composition, the air is subsequently circulated through activated charcoal.

Aspect 34. The method according to any one of Aspect 1-Aspect 33, wherein the terpene is produced by a plant in an enclosed structure, wherein
(a) air from the enclosed structure is circulated through the composition; and
(b) re-circulating the air from step (a) into the enclosed structure.

Aspect 35. The method according to Aspect 34, wherein prior to circulating the air through the composition, heating the air at a temperature of from about 25° C. to about 85° C.

Aspect 36. The method according to Aspect 34 or Aspect 35, wherein after step (a) and prior to step (b), circulating the air through activated charcoal.

Aspect 37. The method according to any one of Aspect 34-Aspect 36, wherein the enclosed structure is a greenhouse, warehouse, storage room, or a processing room.

Aspect 38. A method for reducing the odor produced by a terpene, the method comprising (1) heating the air that comprises the terpene and (2) circulating the air through activated charcoal.

Aspect 39. The method according to Aspect 38, wherein the air is heated at a temperature of from about 25° C. to about 85° C.

Aspect 40. The method according to Aspect 39, wherein the air is warmed by a catalytic converter, a furnace, or a thermal unit.

Aspect 41. The method according to any one of Aspect 38-Aspect 40, wherein the terpene is produced by a plant in an enclosed structure, the method comprising
(a) heating the air from the enclosed structure at a temperature of from about 25° C. to about 85° C.;
(b) circulating the air from step (a) through activated charcoal; and
(c) re-circulating the air from step (b) into the enclosed structure.

Aspect 42. The method according to Aspect 41, wherein the enclosed structure is a greenhouse, warehouse, storage room, or processing room.

Aspect 43. The method according to any one of Aspect 1-Aspect 42, wherein the terpene comprises a hemiterpene, a monoterpene, a sesquiterpene, a diterpene, a sesterpene, a triterpene, a tetraterpene or a polyterpene.

Aspect 44. The method according to any one of Aspect 1-Aspect 42, wherein the terpene is produced from a plant from the genus Cannabis.

Aspect 45. The method according to Aspect 44, wherein the plant is from the species Cannabis sativa, Cannabis indica, or Cannabis ruderalis.

Aspect 46. The method according to Aspect 44, wherein the plant is from the species Cannabis sativa.

Aspect 47. The method according to any one of Aspect 1-Aspect 42, wherein the terpene is produced from the flowers, stems, or leaves of Cannabis sativa.

Aspect 48. The method according to any one of Aspect 1-Aspect 47, wherein after the air has been circulated through the composition, the air is cooled.

From the foregoing, it will be seen that aspects herein are well adapted to attain all the ends and objects hereinabove set forth together with other advantages which are obvious and which are inherent to the structure.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings is to be interpreted as illustrative and not in a limiting sense.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. A method for reducing the odor produced by a terpene, the method comprising circulating air comprising the terpene through (1) a composition comprising an alcohol and a surfactant, (2) a composition comprising an oxidizing agent, or (3) a composition comprising an acid.

2. The method of claim 1, wherein the solution further comprises water.

3. The method of claim 1, wherein the alcohol is an alkyl alcohol, aryl alcohol, alkoxy alcohol, or any combination thereof.

4. The method of any one of claim 1, wherein the alcohol comprises isopropanol and 2-ethyl hexanol, wherein volume ratio of isopropanol to 2-ethyl hexanol is from about 1:0.5 to about 1:4.

5. The method of claim 1, wherein the surfactant comprises a detergent.

6. The method of claim 1, wherein the surfactant comprises sodium lauryl sulfate.

7. The method of claim 1, wherein the oxidizing agent comprises hydrogen peroxide, benzoyl peroxide, t-butyl hydrogen peroxide, di-t-butyl peroxide, t-butyl peroxy benzoate, cumyl hydro peroxide, di-cumyl peroxide, ketone peroxides, a monosulfate, a persulfate, a perborate, a carbonate peroxyhydrate, a phosphate peroxyhydrate, or any combination thereof.

8. The method of claim 1, wherein the oxidizing agent comprises an alkali metal hypochlorite, an alkali metal dichloroisocyanurate, a chlorinated trisodium phosphate, or any combination thereof.

9. The method of claim 1, wherein the oxidizing agent comprises hydrogen peroxide and sodium persulfate.

10. The method of claim 1, wherein the oxidizing agent is up to about 10 dry wt % of the composition.

11. The method of claim 1, wherein the oxidizing agent is two or more agents in the total amount of from about 1 dry wt % to about 5 dry wt % of the composition.

12. The method of claim 1, wherein when the composition comprises an oxidizing agent, the composition further comprises a reducing agent comprising hypophosphorous acid or a salt thereof, a metabisulfite salt, sulfite salt, a bisulfite salt, or any combination thereof.

13. The method of claim 12, wherein the reducing agent is from about 0.5 dry wt % to about 5 dry wt % of the composition.

14. The method of claim 1, wherein the acid comprises a carboxylic acid.

15. The method of claim 1, wherein the acid comprises acetic acid, propionic acid, citric acid, butyric acid, pentanoic acid, hexanoic acid, or any combination thereof in water.

16. The method of claim 1, wherein the concentration of the acid is from about 1 vol % to about 10 vol %.

17. The method of claim 1, wherein the composition further comprises a defoamer, wherein the defoamer comprises a petroleum hydrocarbon, an alcohol, a sulfonated oil, an organic phosphate, a silicone fluid, a dimethylpolysiloxane, a polyalkoxylated polyether, a mineral oil, a vegetable oil, a wax, a hydrophobic silica, a polyoxyethylene-polyoxypropylene copolymer, polyethylene glycol dioleate and propylene glycol monooleate, or any combination thereof.

18. The method of claim 1, wherein prior to circulating the air comprising the terpene through the composition, heating the air at a temperature of from about 25° C. to about 85° C.

19. The method of claim 18, wherein the air is warmed by a catalytic converter, a furnace, or a thermal unit.

20. The method of claim 1, wherein after the air has been circulated through the composition, the air is subsequently circulated through activated charcoal.

21. The method of claim 1, wherein the terpene is produced by a plant in an enclosed structure, wherein
(c) air from the enclosed structure is circulated through the composition; and
(d) re-circulating the air from step (a) into the enclosed structure.

22. The method of claim 21, wherein prior to circulating the air through the composition, heating the air at a temperature of from about 25° C. to about 85° C.

23. The method of claim 21, wherein the enclosed structure is a greenhouse, warehouse, storage room, or a processing room.

24. The method of claim 1, wherein the terpene is produced from a plant from the genus Cannabis.

25. A method for reducing the odor produced by a terpene, the method comprising (1) heating the air that comprises the terpene and (2) circulating the air through activated charcoal.

26. The method of claim 25, wherein the air is heated at a temperature of from about 25° C. to about 85° C.

27. The method of claim 25, wherein the air is warmed by a catalytic converter, a furnace, or a thermal unit.

28. The method of claim 25, wherein the terpene is produced by a plant in an enclosed structure, the method comprising
(d) heating the air from the enclosed structure at a temperature of from about 25° C. to about 85° C.;
(e) circulating the air from step (a) through activated charcoal; and
(f) re-circulating the air from step (b) into the enclosed structure.

29. The method of claim 28, wherein the enclosed structure is a greenhouse, warehouse, storage room, or processing room.
